SEQUENCE LISTING (2 sequences)

SEQ ID NO: 1 - protein, 98 aa, Artificial Sequence
  synthetic peptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 2 - protein, 158 aa, Artificial Sequence
  synthetic peptide
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

(19) United States
(12) Patent Application Publication
(10) Pub. No.: US 2011/0110979 A1
(43) Pub. Date: May 12, 2011

(54) METHOD AND VACCINE FOR OPTIMIZING THE SPECIFIC IMMUNE RESPONSES

(75) Inventor: Denise Nardelli Haefliger, Lausanne (CH)

(73) Assignee: Universite De Lausanne, Lausanne (CH)

(21) Appl. No.: 12/988,455

(22) PCT Filed: Apr. 1, 2009
(86) PCT No.: PCT/IB2009/051372

§ 371 Date: Oct. 18, 2010

Related U.S. Application Data
(60) Provisional application No. 61/071,162, filed on Apr. 16, 2008.

(51) Int. Cl. A61P 35/00; A61K 39/00; A61K 39/12; A61K 39/165; A61K 39/13; A61K 39/29; A61K 39/25; A61K 39/275; A61K 39/09; A61K 39/07; A61K 39/04; A61K 39/112; A61K 39/118; A61P 35/02; A61K 39/145

(52) U.S. Cl. 424/209.1; 424/184.1; 424/204.1; 424/212.1; 424/217.1; 424/218.1; 424/226.1; 424/227.1; 424/230.1; 424/232.1; 424/244.1; 424/246.1; 424/248.1; 424/258.1; 424/263.1; 424/274.1; 424/277.1

(57) ABSTRACT

The present invention relates to a method for treating an infection or disease or lesion, in particular an HPV infection. Furthermore, it relates to a vaccine for treating a Human Papillomavirus (HPV) infection or an associated disease or lesion in a subject.

### CROSS REFERENCE TO RELATED APPLICATIONS

This application is a National Stage filing under 35 U.S.C. §371 of PCT Application No. PCT/IB2009/051372, filed on Apr. 1, 2009. This application also claims the benefit of U.S. Patent Application No. 61/071,162, filed on Apr. 16, 2008. The entirety of both applications is incorporated herein by reference.

### FIELD OF THE INVENTION

The present invention relates to a method for treating an infection or disease or lesion, in particular an HPV infection. Furthermore, it relates to a vaccine for treating a Human Papillomavirus (HPV) infection or an associated disease or lesion in a subject.

### BACKGROUND OF THE INVENTION

Cervical cancer, the second most common cause of cancer death in women worldwide, is the consequence of high-risk types Human Papillomavirus (HPV) infections. The recently developed prophylactic HPV vaccines based on non-infectious L1 Virus-Like Particles (VLPs) have proven to be well tolerated, highly immunogenic and efficient in preventing type specific cervical HPV infection and associated intraepithelial neoplasia (reviewed in Lowy and Schiller, 2006). In contrast, therapeutic vaccines that often target the HPVE6 and/or E7 oncogenes and which are meant to cure already existing HPV-lesions have shown poor clinical efficacy and lack of correlation with the vaccine-specific immune responses measured in peripheral blood (reviewed in Stern, 2005). It is however clear that cell-mediated immune responses are important in controlling both HPV infections and HPV-associated neoplasms as shown by the increased prevalence of these diseases when impaired cell-mediated immunity occurs, such as in transplant recipients or in human immunodeficiency virus-infected patient. The poor efficacy of the experimented therapeutic vaccines also differs from their high pre-clinical efficacy as demonstrated by the regression of ectopic HPV-tumors i.e. TC-1 or C3 cells subcutaneously implanted in the back of mice. In the absence of an animal model of genital infection by a papillomavirus to assess the importance of local cell-mediated immunity for anti-tumor protection, here we have first examined how HPV E7-specific CD8 T cell responses can be induced in the cervico-vaginal (CV) tissue of vaccinated mice. Monitoring of specific T cells responses in the genital mucosa is difficult because of the absence of lymphoid aggregates and the low abundance of T cells in such tissues. Antigen-specific IFN-γ ELISPOT or chromium release cytotoxic assays using genital tissues were rarely performed and required animal pools or large surgical pieces together with in vitro antigen-specific restimulation/expansion steps, which may bias the magnitude and quality of the initial immune response.

For example, US patent application 2004/00115219 (Woong-Shick et al.) disclosed a pharmaceutical composition for prophylaxis and therapy of cell proliferative diseases caused by papillomavirus, comprising an immunologically effective amount of a recombinant E7 papillomavirus antigen and CpG-oligonucleotide administered concomitantly as adjuvant.

An other application, WO2007/137427 (Nventa Biopharmaceuticals corporation) discloses a method of increasing the biological activity of the papillomavirus E7 antigen by administering the recombinant antigen (fused to the HSP protein) along with an immune stimulant. However, these Applicants have shown that a single dose of HSp E7+poly IC followed by three consecutive daily doses of Poly IC alone did not elicit a significant increase in the numbers of E7-specific CD8 cells as compared to a single dose of HspE7+poly IC.

Despite the above mentioned approaches, there is still a need to provide an improved vaccine as well as method for optimizing the specific immune responses.

### SUMMARY OF THE INVENTION

The present invention concerns a vaccine for treating a Human Papillomavirus (HPV) infection or an associated disease or lesion in a subject comprising a complete synthetic E6 and/or E7 polypeptide of an HPV type and an (or combination of) adjuvant.

A further object of the present invention is to provide the use of a HPV vaccine in the treatment of Cervical intraepithelial neoplasia (CIN) type I, II et III, Vulvar intraepithelial neoplasia (VIN) I, II III, Vaginal IN, I,II II, Condyloma, Anal IN, I, II, III, genital warts, ano-genital cancer and non genital HPV-related lesions.

The invention also contemplates a method for treating an infection or disease or lesion in a subject comprising i) administering a vaccine comprising an antigen, and ii) at a determined time, subsequently applying one or several consecutive dose of an immunostimulant at a site where the infection, the lesion or a disease is present.

Another object of the present invention concerns a method for treating a Human Papillomavirus infection or a related disease or lesion in a subject comprising i) administering the vaccine of the invention, or any vaccine suitable for treating a Human Papillomavirus (HPV) and, ii) at a determined time, subsequently applying one or several consecutive dose of an immunostimulant at a site where the Human Papillomavirus infection, the related disease or the lesion is located.

### DESCRIPTION OF THE FIGURES

FIG. 1. shows the E7-specific CD8 T cells responses in PBMC after sc (A) or i.n. (B) immunization with E7₁₋₉₈.

C57BL/6 mice were immunized sc (A) or i.n. (B) as indicated below each graph. The E7₄₉₋₅₇-specific CD8 T cell responses were analyzed in PBMC by ex vivo IFN-γ ELISPOT as detailed in the Materials and Methods section. Results are shown as the number of E7-specific IFN-γ secreting cells/10⁵ PBMC. The horizontal bars represent the mean response of each group of mice. Groups of mice immunized with E7₁₋₉₈+adjuvants that significantly differed from the group of mice that received E7₁₋₉₈ alone are indicated with * for p<0.05 and ** for p<0.01 (Dunnet's post test of one way ANOVA).

FIG. 2. shows the kinetics of E7-specific CD8 T cell response in PBMC, spleen and CV tissue after s.c. or i.n. vaccination

C57BL/6 mice were immunized with E7₁₋₉₈+HLT and CpG or +HLT and R848 by sc (left side graphs) and i.n. (right side graphs) routes, respectively. At days 7, 9 and 11 mice were sacrificed and the E7₄₉₋₅₇-specific CD8 T cell responses were analyzed in PBMC, spleen and CV tissue by ex vivo IFN-γ ELISPOT as detailed in the Materials and Methods section. Results are shown as the number of E7-specific IFN-γ secreting cells/10⁵ cells. The horizontal bar represents the mean response of mice at each time point. Results were compared within time points and between immunization routes by a Student's t test. Significant differences between groups are indicated with * for p<0.05 and ** for p<0.01

FIG. 3. shows the lack of correlation between E7-specific CD8 T cell responses obtained in PBMC, spleen and CV tissues of the same mice after sc or i.n. vaccination.

E7-specific CD8 T cell responses of 20 E7₁₋₉₈ sc immunized mice (A, C and E) or 23 E7₁₋₉₈ i.n. immunized mice (B, D and E) were examined in PBMC, splenocytes and CV tissues. Results of CV tissues (vertical axis) were plotted against the results in PBMC (horizontal axis (A and B) or in splenocytes (horizontal axis in C and D) from the same animals. Results of splenocytes (vertical axis) were plotted against the results in PBMC (horizontal axis in E and F) from the same animals. Correlation r by Pearson and p values are indicated.

FIG. 4. shows the anti-tumor effect of sc or i.n. immunization with the E7 vaccine

Three groups of 9 C57BL/6 mice were immunized sc with PBS (control), sc with E7₁₋₉₈+HLT and CpG or i.n. E7₁₋₉₈+CpG, HLT and R848. Six days later, C57BL/6 mice were challenged sc in their flank with 2×10⁴ TC-1 cells. The mean tumor volumes±SEM of the three groups of mice are shown in (A) and % of tumor free mice in each group are shown in (B).

FIG. 5. shows the E7-specific CD8 T cells responses in CV tissue and PBMC after sc immunization with E7₁₋₉₈ and ivag treatment with immunostimulants

C57BL/6 mice in diestrus stage of their estrus cycle were immunized sc with E7₁₋₉₈+HLT and CpG. At day 6 the immunostimulants indicated below each graph were ivag administered. At day 9, the E7₄₉₋₅₇-specific CD8 T cell responses were analyzed in CV tissue (A) and PBMC (B) by ex vivo IFN-γ ELISPOT. Results are shown as the mean±SEM number of E7-specific IFN-γ secreting cells/10⁵ CV or PBMC of each group of mice. Groups of mice that received ivag immunostimulants that significantly differed from the group of mice that received ivag PBS are indicated with * for p<0.05, ** for p<0.01 and *** for p<0.001 (Dunnet's post test of one way ANOVA).

FIG. 6. shows the E7 specific CD8 T cell responses in CV tissue and PBMC after sc immunization with E7₁₋₉₈ followed by ivag live attenuated Salmonella treatment

C57BL/6 mice in diestrus stage of their estrus cycle were immunized sc with E7₁₋₉₈+HLT and CpG. At day 6 ca. 10⁹ CFU of attenuated live AroA or PhoPc bacteria, heat-killed AroA or recombinant live E7 expressing PhoPc (PhoP^{c}E7) were ivag administered as indicated below each graph. At day 9, the E7₄₉₋₅₇-specific CD8 T cell responses were analyzed in CV tissue (A and C) and PBMC (B) by ex vivo IFN-γ ELISPOT. Results are shown as the mean±SEM number of E7-specific IFN-γ secreting cells/10⁵ CV or PBMC of each group of mice. Groups of mice that received ivag immunostimulants that significantly differed from the group of mice that received ivag PBS are indicated with * for p<0.05, ** for p<0.01 and *** for p<0.001

FIG. 7. shows the E7-specific CD8 T cell responses in CV tissue and PBMC after i.n. immunization with E7₁₋₉₈ and ivag treatment with CpG.

C57BL/6 mice in diestrus stage of their estrus cycle were immunized i.n. with E7₁₋₉₈+HLT, R848 and CpG. At day 6 the mice received an ivag treatment with CpG or PBS. At day 9, the E7₄₉₋₅₇-specific CD8 T cell responses were analyzed in CV tissue (A) and PBMC (B) by ex vivo IFN-γ ELISPOT. Results are shown as the mean±SEM number of E7-specific IFN-γ secreting cells/10⁵ CV or PBMC of each group of mice. Groups of mice that received ivag immunostimulants that significantly differed from the group of mice that received ivag PBS are indicated with * for p<0.05, ** for p<0.01 and *** for p<0.001

FIG. 8. shows the effect of ivag treatment with CpG on the E7-specific CD8 T cell responses in CV tissue and PBMC.

C57BL/6 mice in diestrus stage of their estrus cycle were immunized sc with E7₁₋₉₈+HLT and CpG. Ivag CpG were administered once at day 6 (A and B) or three times at day 6, 9 and 12 (C). At day 15, the E7₄₉₋₅₇-specific CD8 T cell responses were analyzed in CV tissue (A and C) and PBMC (B) by ex vivo IFN-γ ELISPOT. Results are shown as the mean±SEM number of E7-specific IFN-γ secreting cells/10⁵ CV or PBMC of each group of mice. Groups of mice that received ivag immunostimulants that significantly differed from the group of mice that received ivag PBS are indicated with * for p<0.05, ** for p<0.01 and *** for p<0.001

FIG. 9. shows the anti-tumor effect of a topical immunostimulant after sc immunization with the E7 vaccine.

C57BL/6 mice in diestrus stage of their estrus cycle were challenged sc in their flank with 2×10⁴ TC-1 cells at day 1. At day 9, mice received sc E7 vaccine or PBS. At day 14, one group of E7 vaccinated mice and one group of PBS vaccinated mice receive 30 μg CpG sc next to their tumors. The mean tumor volumes±SEM of the four groups of mice are shown in (A), individual tumor volume of individual mice at day 26 for the four groups of mice (C) and at day 36 for the two groups of E7-vaccinated mice (D) are also shown the horizontal bar indicating the mean value for each mice group. % of tumor free mice in each group is shown in (B).

FIG. 10. shows that E7-specific cytotoxic T cells are induced in vivo in vaccinated mice.

C57/B16 mice were sc vaccinated with E7₁₋₉₈+HLT and CpG and 7 days later were i.v. transferred with 10⁷ CFSE^{high} labeled E7₄₉₋₅₇-pulsed splenocytes and 107 CFSE^{low} labeled unpulsed splenocytes. Individual spleen and Genital LN harvested 15 days later were analyzed by flow-cytometry for the presence of CFSE^{high} and CFSE^{low} cells. Representative results from one mouse are shown in A (spleen) and B (Genital LN). The means % of cell lysis±SEM (see material and method for calculation of % lysis) in spleen (black bars) and Genital LN (grey bars) are indicated in C.

FIG. 11. shows that high avidity E7-specific IFN-γ secreting CD8 T cells are induced in cervico-vaginal tissue after E7 vaccination.

Five C57/B16 mice were s.c. vaccinated with E7₁₋₉₈+HLT and CpG and 8 days later their PBMC, spleen, Inguinal and Genital LN as well as CV (as indicated below the bars) were assayed by IFN-γ ELISPOT using log dilutions of the E7₄₉₋₅₇ peptide. The mean±SEM Ratio between high and high+low avidity E7-specific CTL (i.e. activated with 10⁻¹⁰ M and 10⁻⁶ M E7 peptide, respectively) are shown.

FIG. 12. shows the effect of intravesical instillation of immunostimulant (CpG) on the E7-specific CD8 T cell response in the bladder.

Mice received the sc E7 vaccine (as a model vaccine) followed 5 days later by a 100 μg dose of CpG instillated intravesically through a catheter inserted into the urethra. The E7-specific CD8 T cells responses were measured by IFN-γ ELISPOT in spleen and bladder three days later.

FIG. 13. shows the anti-tumor effect of an ivag immunostimulant after sc immunization with the E7 vaccine in a genital tumor protection assay

Engineered TC-1 cells that express the luciferase gene (TC-1-luc) so that the intravaginal tumors can be followed by in vivo bioluminescence imaging after i.p. injection of luciferine with a Xenogen camera. % of tumor take 12 days after ivag instillation of 12′500 TC-1-luc cells (A). Three groups of 5 mice harboring ivag TC-1-luc tumors were compared: two groups of mice received 12 days after TC-1-luc ivag administration one dose of the s.c. E7₁₋₉₈+CpG+HLT vaccine either alone or followed by an ivag instillation of 100 ng CpG, while the third group was left unvaccinated (B)

### DETAILED DESCRIPTION OF THE INVENTION

The present invention provides a vaccine for treating a Human Papillomavirus (HPV) infection or an associated disease or lesion in a subject comprising a complete synthetic E6 and/or E7 polypeptide of an HPV type and an adjuvant.

As used herein, “a” or “an” means “at least one” or “one or more.”

The terms “peptide”, “protein”, “polypeptide”, “polypeptidic” and “peptidic” are used interchangeably to designate a series of amino acid residues connected to the other by peptide bonds between the alpha-amino and carboxy groups of adjacent residues.

“An antigen” is a molecule that may stimulate an immune response. As used herein, antigens are usually proteins or polysaccharides or nucleic acids of, for example, an HPV. This includes parts (coats, capsules, cell walls, flagella, fimbrae, and toxins) of bacteria, viruses, and other microorganisms. These proteins, polysaccharides, or nucleic acids used to stimulate an immune response are present either on live or dead organism and can be used as such.

The terms “treating” as used herein refers to therapeutic treatment.

“Administering”, as it applies in the present invention, refers to contact of a therapeutically effective amount of the vaccine of the invention, to the subject, preferably a woman or a man.

The term “a (papillomavirus)-associated disease” denotes cell-proliferative disease of malignant or nonmalignant cell populations caused by papillomavirus, which morphologically often appear to differ from surrounding tissues.

The papillomavirus protein, which can be comprised in vaccine of this invention and prepared by protein synthesis methods, denotes the protein that has the complete sequence of natural protein as well as 85% or more, preferably 90% or more, of sequence homology and induces the substantially same immune response as that of the natural complete papillomavirus protein.

The particularly preferred HPV protein of this invention is the complete E7 polypeptide of an HPV (E7₁₋₉₈) of protein of human papillomavirus type 16. The E7 protein is a small (approximately 10,000 Mw), with the following polypeptidic sequence:

MHGDTPTLHEYMLDLQPETTDLYCYEQLNDSSEEEDEIDGPAGQAEPD
RAHYNIVTFCCKCDSTLRLCVQSTHVDIRTLEDLLMGTLGIVCPICSQ
KP.

The E6 protein is larger and has the following polypeptidic sequence:

MHQKRTAMFQDPQERPRKLPQLCTELQTTIHDIILECVYCKQQLLRRE
VYDFAFRDLCIVYRDGNPYAVCDKCLKFYSKISEYRHYCYSLYGTTLE
QQYNKPLCDLLIRCINCQKPLCPEEKQRHLDKKQRFHNIRGRWTGRCM
SCCRSSRTRRETQL

E7 is a Zn-binding phosphoprotein that has oncogenic properties, likely due to its ability to bind to the retinoblastoma gene product Rb (a tumor suppressor binding to and inactivating transcription factor E2F). The transcription factor E2F controls transcription of a number of growth-related genes including those encoding thymidine kinase, c-myc, dihydrofolate reductase and DNA polymerase alpha. Rb-E2F complex formation prevents the expression of the latter genes in GO and G1 phases, restricting their expression to the S phase where the Rb-E2F complexes are programmed to dissociate, liberating active transcription factor E2F. Thus E7 represents an attractive target for immunological intervention in papilloma virus infections as it is expressed throughout the virus lifecycle and indeed it is one of only two viral proteins expressed during late stage cervical carcinoma caused by HPV infection. Preferably also the vaccine comprises an E6 and/or E7 synthetic polypeptide as antigens. These polypeptides may be free in the composition of fused together so as to obtain a unique polypeptide comprising the two sequences or each fused to a polypeptides having intrinsic adjuvant properties (such as HSP70 . . . ).

The complete E7₁₋₉₈ and E6₁₋₁₅₈ proteins used in this invention can be prepared by various protein synthesis known in the art. Alternatively the complete E7₁₋₉₈ protein of the invention may be prepared to include D-forms and/or “retro-inverso isomers” of the peptide.

Usually, the E6 and/or E7 polypeptide of an HPV type are synthetic, i.e. prepared by protein synthesis. The use of polypeptides from biological sources, either natural sources or recombinant polypeptides expressed in a host system, allows for the use of both small and larger polypeptides, either in purified form or as crude preps for vaccination purposes and is routinely applied in the art of vaccination. However, the use of polypeptides or recombinant polypeptides from biological sources requires extensive purification and quality control. Inherently the production of polypeptides or recombinant polypeptides from biological sources is subject to biological variations, various contaminants and errors. Because of the inherent variability and unpredictability of biological sources, the high rate of mutations and epigenetic changes in cell lines, bacteria, viruses and vectors used, the threat of contamination with DNA, in particular viral or recombinant DNA, the safety and quality control requirements set by regulatory authorities such as the EMEA (European Medicines Evaluation Agency), the US FDA (Food and Drug Administration) or the Japanese Pharmaceutical and Food Safety Bureau of Ministry of Health, Labour and Welfare are extensive and extremely strict. Clinical validation and approval of preparations for vaccination by the medical authorities and mandatory use of GMP grade materials, equipment and procedures make the use of recombinant polypeptides from biological sources extremely laborious, risky, costly and generally unattractive.

“Adjuvants” are pharmacological or immunological agents that modify the effect of a vaccine while having few if any direct effects when given by themselves. Preferably, the adjuvant is added at a dosage of about 0.01 μg/dose of vaccine to about 20 mg/dose of vaccine, more preferably 0.1 μg/dose of vaccine to about 75 μg/dose of vaccine depending on the type of adjuvant and the subject. The dose of vaccine and adjuvant will depend on various factors such as weight, age, sex, administration route, formulation, time, and the general health condition, etc of individuals.

Preferably the adjuvant of the invention is selected from the group comprising a bacterial toxin, a toll-like receptor (TLR) agonist, or a combination thereof.

Toll-like receptors (TLRs) are a class of single membrane-spanning non-catalytic receptors that recognize structurally conserved molecules derived from microbes once they have breached physical barriers such as the skin or intestinal tract mucosa, and activate immune cell responses. They are believed to play a key role in the innate immune system.

Non-limiting examples of TLR2 agonists comprise synthetic triacylated and diacylated lipopeptides. An exemplary, non-limiting TLR2 ligand is Pam3Cys (tripalmitoyl-S-glyceryl cysteine) or S-[2,3-bis(palmitoyloxy)-(2R5)-propyl]-N-palmitoyl-(R)-cysteine, where “Pam3” is “tripalmitoyl-S-glyceryl”). Aliprantis et al. (1999) Science 285: 736-739. Derivatives of Pam3Cys are also suitable TLR2 agonists, where derivatives include, but are not limited to, S-[2,3-bis(palmitoyloxy)-(2-R, 5)-propyl]-N-palmitoyl-(R)-Cys-(S)-Ser-Lys4-hydroxytrihydrochloride; Pam3Cys-Ser-Ser-Asn-Ala; Pam3Cys-Ser-(Lys) 4; Pam3Cys-Ala-Gly; Pam3Cys-Ser-Gly; Pam3Cys-Ser; Pam3Cys-OMe; Pam3Cys-OH; PamCAG, palmitoyl-Cys ((RS)-2,3-di (palmitoyloxy)-propyl)-Ala-Gly-OH; and the like. Another non-limiting example of a suitable TLR2 agonist is PAM2CSK4. PAM2CSK4 (dipalmitoyl-S-glyceryl cysteine-serine-(lysine)₄; or Pam2Cys-Ser-(Lys) 4) is a synthetic diacylated lipopeptide, Muramyl dipeptide (MDP) (N-acetylmuramyl-L-alanyl-D-isoglutamine) is derived from the cell wall of mycobacteria and is one of the active components in the Freund complete adjuvant.

Non-limiting examples of TLR4 agonists: LPS well known TLR-4 agonist is itself only experimentally been used as an adjuvant due to its high toxicity Several studies have been conducted with chemically modified forms of lipid A. One of the best studied is monophosphoryl lipid A (MPL). MPL is derived from the LPS of Salmonella minnesota. Like LPS, MPL is thought to act via TLR4 and TLR-2.

A non-limiting example of TLR5 agonists is flagellin.

Non-limiting examples of TLR7/8 agonists comprise the ssRNA sequences ssRNA8A and ssRNA40 (these sequences were previously identified as TLR7- and TLR8-specific agonists, respectively) and small purine-like molecules such as imidazoquinoline (imiquimod, resiquimod).

Non-limiting examples of TLR9 agonists comprise Cytidine-phosphate-Guanosines (CpG). CpG are unmethylated dinucleotides present at a frequency of approximately 1 on 16 nucleotides in bacterial DNA, whereas they are underrepresented (1/50 to 1/60) and methylated in the vertebrate (mammalian) genomes. Because of these differences, a nonself pattern recognition mechanism has evolved in the vertebrate immune system using PRR enabling them to encounter invading pathogens via their unmethylated CpG-dinucleotides. The biological activity of these CpG dinucleotides can be mimicked by chemically synthesized CpG-oligodeoxynucleotides (CpG-ODN). CpG-ODN are chemically synthesized single stranded DNA sequences that are able to stimulate MØ, NK cells, DC and B cells. They were originally synthesized in a specific motif in which the CpG-dinucleotide is flanked preferentially by two purines, adenine (A) or guanine (G) at the 5′-end, and two pyrimidines, cytosine (C) or thymine (T) at the 3′-end, making for example AGCpGTT. Examples of optimized CpG-ODN are from Coley Pharmaceutical group, (CpG ODN 1826 for use in mice, CpG-ODN 7909 for use in human).

One of the problems with the oral immunization is that the CpG-ODN are rapidly degraded in the gastrointestinal tract. Therefore a synthesized oligonucleotides consisting of a novel 3′-3′-linked structure and synthetic stimulatory motifs now exists and is called second-generation immunomodulatory oligonucleotides or IMO. These IMO were more stable in the murine gastrointestinal tract resulting in a stronger immune response, making them a potentially interesting intestinal adjuvant.

Synthetic TLRs agonists have been described in the literature (Lee et al. (2003) and references therein whose teaching is incorporated herein).

TLRs can also form heterodimers having unique ligand specificities. For example, the macrophage-activating lipopeptide 2 (MALP-2) from mycoplasma is a ligand for TLR2/TLR6 heterodimers whereas the bacterial lipopeptide Pam3Cys-Ser-Lys(4) is a ligand for TLR1/TLR2 heterodimers.

Usually the bacterial toxin is selected from the group comprising ADP-ribosylating enterotoxins (cholera toxin (CT) and the heat-labile enterotoxin of Escherichia coli: LT-I, LT-IIa and LT-IIb are potent systemic and mucosal adjuvants (review in Freytag and Clements, 2005). Both LT and CT are synthesized as multisubunit toxins with A and B components. The A-subunit is the enzymatically active moiety and consists of two chains, A1and A2, joined by a proteolytically sensitive peptide (Arg192) subtended by a disulfide loop. Like other A-B bacterial toxins, LT and CT require nicking and disulfide reduction to be fully biologically active. When LT or CT first encounter a mammalian cell, they bind to the surface through interaction of the B-subunit pentamer. The principle receptor for both LTB and CT-B is GM1-ganglioside, a glycosphingolipid found ubiquitously on the surface of mammalian cells. A principal effect of the B-subunit interaction with mammalian cells is the stable cross-linking of GM1 at the cell surface. The adjuvant effect was determined to be a function of the enzymatically active A-subunit of the toxin.

The two active site mutations that have been most extensively characterized are lysine for serine at position 63 (LT(S63K)) and arginine for alanine at position 72 (LT(A72R)). These two mutations differ in the amount of residual enzymatic activity each possesses, with LT(A72R) retaining a higher level of enzyme and adjuvant activity.

An alternative approach to detoxification of LT was construct a mutant of LT containing a single amino acid substitution altering the site of proteolytic cleavage within the disulfide subtended region joining A1 and A2: Arg at position 192 to Gly (LT(R192G)).

A nontoxigenic mutant of heat-labile enterotoxin (LT) from Escherichia coli, LTK63, has proven to be safe and a potent mucosal adjuvant in animals following intranasal administration. Remarkably, recent phase I clinical trials of a trivalent inactivated influenza vaccine delivered with LTK63 have demonstrated its safety also in humans (Stephenson et al. 2006).

Also envisioned in the present invention is the co-administration of the vaccine with a combination of several adjuvants like requisimod, the heat-labile enterotoxin HTL and CpG ODN.

The vaccine can be administered at a dosage of about 0.1 μg/kg/day to about 3 μg/kg/day, preferably 0.5 μg/kg/day to about 1 μg/kg/day at an interval which will depend on the route of administration. It depends on various factors such as weight, age, sex, administration route, formulation, time, and the general health condition, etc of individuals.

Over 100 different HPV types have been identified and are referred to by number. For example, types 6, 11 may cause genital warts whereas types 16, 18, 31, 33, 35, 39, 45, 51, 52, 56, 58, 59, and 68 are “high-risk” sexually transmitted HPVs and may lead to the development of HPV infection associated disease or lesion selected from the group comprising Cervical intraepithelial neoplasia (CIN) type I, II et III, Vulvar intraepithelial neoplasia (VIN) I, II III, Vaginal IN, I,II II, Condyloma, Anal IN, I, II, III, genital warts, ano-genital cancer and non genital HPV-related lesions.

The use of the vaccine of invention in the treatment of Cervical intraepithelial neoplasia (CIN) type I, II et III, Vulvar intraepithelial neoplasia (YIN) I, II III, Vaginal IN, I,II II, Condyloma, Anal IN, I, II, III, genital warts, ano-genital cancer and non genital HPV-related lesions is also contemplated.

The vaccine can be administered mucosally. Mucosal administration is advantageous to induce mucosal immune responses in addition to the systemic one. Mucosal administration may include (not limited to) oral, intranasal, aerosol, rectal or vaginal administration. The preparations for mucosal administration include transdermal devices, aerosols, creams, lotions or powders pending on the mucosal site. Preferably, the mucosal administration is intranasal.

The vaccine of the invention can be formulated with one or more pharmaceutically acceptable carrier (in addition to the adjuvants listed above) that facilitates the formulation, including excipients. The formulation depends on the administration route. For injection, the vaccine can be formulated into aqueous solutions, preferably in a saline solution. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. For administration by inhalation, the active ingredient is conveniently delivered in the form of an aerosol spray presentation from pressurized packs or a nebuliser with the use of a suitable propellant or the form of a powder, which can be formulated into cartridges. Also, when administered by injection, the vaccine can be formulated into forms such as suspensions, solutions, emulsions, etc.

The invention also provides a method for treating an infection or disease or lesion in a subject comprising

i) administering a vaccine comprising an antigen, and

ii) at a determined time, subsequently applying one or several consecutive dose of an immunostimulant at a site where the infection, the lesion or a disease is present.

Applicants have shown that it is possible to enhance the therapeutic properties of a vaccine by, after having administered said vaccine, subsequently and at a determined time applying one or several consecutive dose of an immunostimulant at a site where an infection, a lesion or a disease is present.

The aims of this method are

(i) to attract and/or activate additional vaccine-specific T cells that have been induced by the prime vaccination during the first step of the method of the invention,

(ii) to induce a microenvironment of inflammatory cytokines and chemokines that relieve local immunosuppressive status and thus promote tumor regression.

This method is applicable to any kind of vaccines and antigens for treating or preventing a disease. Non-limiting examples of diseases selected are from the group comprising Anthrax, Candida, Cervical Cancer (Human Papillomavirus), Chlamidia, Diphtheria, Hepatitis A, Hepatitis B, Haemophilus influenzae type b (Hib), Human Papillomavirus (HPV), HIV, Influenza (Flu), Japanese encephalitis (JE), Lyme disease, Measles, Meningococcal, Monkeypox, Mumps Pertussis, Pneumococcal, Polio, Rabies, Rotavirus, Rubella, Shingles (Herpes Zoster), HSV, Smallpox, Tetanus, Typhoid, Tuberculosis (TB), Varicella (Chickenpox) and Yellow Fever.

In case the vaccine comprises a tumor-antigen, then the diseases are selected from the non-limiting cancer group comprising melanoma, colon cancer, bladder cancer, breast cancer, prostate cancer, lung cancer carcinoma, lymphoma, blastoma, sarcoma, liposarcoma, neuroendocrine tumor, mesothelioma, schwanoma, meningioma, adenocarcinoma, leukemia, lymphoid malignancy, squamous cell cancer, epithelial squamous cell cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, hepatoma, rectal cancer, colorectal cancer, endometrial or uterine carcinoma, salivary gland carcinoma, kidney or renal cancer, vulval cancer, thyroid cancer, hepatic carcinoma, anal carcinoma, penile carcinoma, testicular cancer, esophageal cancer, a tumor of the biliary tract, and head and neck cancer.

Usually, the “determined time” corresponds to the time or moment just before where the vaccine-specific T cell response (CD4 and/or CD8) is high comparing to the response without vaccine activation. Preferably, the vaccine specific T cell response is a CD8 T cell response.

This determined time depends on various factors such as the vaccine, the administration route, the formulation, the general health condition, the subject. However, this time is generally comprised between 4 to 11 days after administering the vaccine, preferably between 4 to 9 days, more preferably between 4 to 8 days and even more preferably 6-8 days after administering the vaccine. Typically, this is determined by analysis of the presence of vaccine-specific T cells (tetramer staining) or activity of vaccine-specific T cells (CD4 T cell proliferation, IFN-γ secretion, IFN-γ ELISPOT, IFN-γ intracellular staining or CTL activity) in blood or tissue samples as described herein.

Also envisioned in the present method is the fact that steps i) and ii) can be repeated as often as necessary in order to enhance the immune response and the efficacy of the treatment.

In case the disease to be treated according to the invention is an HPV related disease or lesion, then the method for treating a Human Papillomavirus infection or a related disease or lesion in a subject comprises

i) administering the vaccine as described above, or any vaccine suitable for treating a Human Papillomavirus (HPV) and,

ii) at a determined time, subsequently applying one or several consecutive dose of an immunostimulant at a site where the Human Papillomavirus infection, the related disease or the lesion is located.

As described above, the immunostimulant is administered topically. Topical administration is advantageous so as to localize the immunostimulant in the site administered, with minimized systemic uptake. When administered topically, smaller dosages than other administration routes can be administered. The preparations for topical administration include transdermal devices, injections, aerosols, creams, lotions, powders, etc.

Usually, the Human Papillomavirus infection related disease is selected from the group comprising Cervical intraepithelial neoplasia (CIN) type I, II et III, Vulvar IN I, II III, Vulvar intraepithelial neoplasia (VIN) I,II II, Condyloma, Anal IN, I, II, III, genital warts, ano-genital cancer and non genital HPV-related lesions.

In the method described above, the at least one “immunostimulant” of step ii) is usually a radiation or an agent able to recruit immune effectors cells and/or relieve local immunosuppressive status via the induction of local cytokines and chemokines.

Preferably, the agent able to recruit immune effectors cells and/or relieve local immunosuppressive status is selected from the group comprising TLR-agonists, pro-inflammatory molecules and live attenuated bacterial or viral vaccines strains. The immune stimulant is preferably applied at an amount from about 0.1 μg to about 200 mg or between 10⁵ and 10¹¹ CFU or PFU (for live bacterial or viral vaccine strains), or any amount there between. Where the live bacterial or viral vaccine strain used as immunostimulant in the method (ii) is recombinant and express the antigen included in the vaccine (i) then the live recombinant bacterial or viral vaccine strain can act both as an immunostimulant and as a vaccine booster. Preferred recombinant live bacterial vaccine strain (as immunostimulant) is an attenuated Salmonella expressing E7 of HPV16.

As already described above, the toll-like receptor agonist is selected from the group comprising an agonist of TLR 2, TLR 3, TLR 4, TLR 5, TLR 7/8 or TLR 9.

It is also envisioned that in some cases the immunostimulant of the method and the adjuvant of the vaccine are the same.

Generally, the pro-inflammatory molecule of the method of the invention is selected from the group comprising detergent, spermicide (nonoxynol-9), microbicide, cytokines, whereas the live bacterial or viral vaccine strains is selected from the group comprising live attenuated Salmonelle enterica serovar Typhimurium and Typhi strain, Bacille Calmette Guérin (BCG) strains, live attenuated Listeria monocytogenes strains, Lactococcus lactis strain, Streptococcus gordonii strain, attenuated Candida strains, attenuated Chlamydia strains, Modified vaccinia Ankara (MVA) strains and Nyvac strains and FMSE (Tick-borne encephalitis virus vaccine strains).

Usually, the cytokines include granulocyte-macrophage colony stimulating factor (GM-CSF), granulocyte colony stimulating factor (GCSF), IL-1, IL-2, IL-3, IL-4, IL-5, IL-6, IL-7, IL-10, IL-12, IL-15, TNF-[alpha], TNF-[gamma], Flt3 ligand, etc.

As described above, the determined time is the time just before the peak in the vaccine specific T cell response. Usually, the vaccine specific T cell response is a CD8 T cell response and the determined time is usually comprised between 3 days to one minute before the peak in the vaccine specific T cell response.

Preferably, the time where there is a peak in the vaccine specific CD8 T cell response is generally comprised between 4 to 11 days after administering the vaccine, preferably between 4 to 9 days, more preferably between 4 to 8 days and even more preferably 5-7 days after administering the vaccine of the invention or any other HPV vaccine.

The site corresponds to the region of the body where the Human Papillomavirus infection, the related disease or the lesion is located. For example in case the HPV infection related disease is Cervical intraepithelial neoplasia (CIN) type I, II et III then the site corresponds to the genital mucosa.

When the method of the invention comprises, in step i), administering any vaccine suitable for treating a Human Papillomavirus (HPV), then the HPV type is preferably selected from the group consisting of: HPV6, HPV11, HPV16, HPV18, HPV26, HPV31, HPV33, HPV35, HPV39, HPV45, HPV51, HPV52, HPV53, HPV55, HPV56, HPV58, HPV59, HPV66, HPV68, HPV73, and HPV82.

Also envisioned in the present method is the fact that steps i) and ii) can be repeated as often as necessary in order to enhance the immune response and the efficacy of the treatment.

The foregoing description will be more fully understood with reference to the following Examples. Such Examples, are, however, exemplary of methods of practicing the present invention and are not intended to limit the scope of the invention.

### EXAMPLES

### Example 1

### HPV16 E7-Specific CD8 T Cell Immune Responses in the Genital Mucosa of Vaccinated Mice

Materials and Methods

### 1. Immunization of Mice

Six to eight-week-old female C57BL/6 mice (Iffa Credo, France) were used in all experiments following ethical directives of the Swiss veterinary authorities. An E7₁₋₉₈ polypeptide synthetized by Protein and Peptide Chemistry Facility of the Institute of Biochemistry (UNIL. Switzerland) was administered sc in the back at the base of the tail or in the neck, as well as by the i.n. route in anesthetized mice (Balmelli et al., 2002). The following adjuvant were co-administered alone or in combinations as mentioned in the results sections: Resiquimod (R-848, Huang Lisheng Pharmatec, China, 75 μg/dose sc or i.n.), the heat-labile enterotoxin HLT (0.4 μg/dose sc. or 10 μg/dose i.n.), derived from a natural non-toxic variant of E. coli (Gluck et al., 2000) and CpG ODN 1826 optimized for stimulation of the mouse immune system (Coley Pharmaceutical Group, Wellesley, Mass. Groups, 10 μg/dose sc or i.n.).

### 2. Preparation of Splenocytes, Lymph Nodes (LN) and CV Suspensions

Mice were sacrificed by inhalation of CO₂ and spleen, LN and genital tract were harvested. Single-cell suspensions were obtained by pressing the spleen and LN onto a 70 μm filter (Falcon) using a syringe piston and subsequently passing the cells through a 40 μm filter (Falcon). Dissociated cells were resuspended in complete high-glucose Dulbecco's modified Eagle medium (DMEM, containing glutamax-1 and sodium pyruvate supplemented with 10 mM HEPES, 1× non-essential amino acids, 100 U of penicillin-streptomycin/ml, 10% fetal calf serum (FCS), all from Invitrogen, and 20 μM 2-mercaptoethanoll, Sigma). Cell viability was determined by trypan blue exclusion.

The CV tissues were minced and washed twice in Extraction buffer (HBSS and 10 mM dithiotreitol, DTT). Minced tissues were then digested with 0.5 mg/ml thermolysin (Roche) in extraction buffer for 45 minutes at 4° C. under agitation and then filtered through 150 μm-pore-diameter nylon filters. Isolated cells were kept at 4° C., and the remaining tissues were digested with 1 mg/ml collagenase/dispase (Roche) in IMDM with glutamax-1 (Gibco, Invitrogen) supplemented with 20% FCS for 45 minutes at 37° under agitation and filtered as described above. Cells isolated after both enzymatic digestions were pooled, layered onto FCS and centrifuged for 20 minutes at 1′800 rpm without brake. Pelleted cells were then digested with 2 mg/ml DNase (Sigma) for 30 minutes at 37° C. under agitation and filtered through 50 μm-pore-diameter nylon filters. Cells were collected after centrifugation and counted. Typically, between 2×10⁵-1×10⁶ cells could be isolated from the vagina/cervix, depending on the estrous cycle stage of the mice.

### 3. Preparation of Peripheral Blood Monocytes (PBMC)

100-120 μl of tail blood is collected in 250 μl of PBS containing liquemin (500UI/ml). PBMC were purified on lympholyte M (Cedarlane CL 5035) gradients and resuspended in complete DMEM medium after red blood cell lysis.

### 4. IFN-γ ELISPOT Assay

Multiscreen-HA 96-well plates (MAHA 54510, Millipore) were coated overnight at 4° C. with a monoclonal anti-IFN-γ antibody (R4-6A2, Pharmingen) at a concentration of 10 μg/ml in PBS. Plates were then blocked during 2 hours at 37° C. with PBS/BSA 1%. 20000 to 100000 cells/well were incubated in duplicate with 1 μg/ml of the E7₄₉₋₅₇ peptide or medium alone (control wells) during 16-24 hours in the 96-well ELISPOT plates. Then, a biotinylated monoclonal anti-IFN-γ antibody (AN 18.03.C12 (16)) was added at a concentration of 2 μg/ml in PBS/BSA 1% and plates were incubated for 2 hours at 37° C.: between each incubation step, plates were washed sex times with PBS/Tween-20 0.1% (PBT). After 1 hour incubation with streptavidin-alkaline phosphatase conjugate (1/2000 in PBT, Boehringer), plates were developed with a solution of BCIP/NBT (Roche) until apparition of blue spots. Tap water was used to stop the reaction and the plates were dried in air overnight. Individual spots were counted under a dissecting microscope. E7-specific responses are defined for each individual mouse as the number of IFN-γ spots/10⁵ cells in the E7-stimulated wells−the number of IFN-γ spots/10⁵ cells in the control wells. The limit of detection was calculated to be higher than the mean+3 SD of the E7-specific responses of 5 naïve mice, and corresponded to 3 spots/10⁵ cells in LN samples, 2 spots/10⁵ cells in PBMC and 2 spots/10⁵ cells in CV samples
5. Challenge of Mice with Tumor Cells
The TC-1 cell line was generated by transduction of C57BL/6 primary lung epithelial cells with a retroviral vector expressing HPV16 E6/E7 plus a retrovirus expressing activated c-Ha-ras. These cells (kindly provided by Prof. T-C Wu, Johns Hopkins Medical Institutions, Baltimore, USA) were cultured in RPMI 1640+glutamax-1 supplemented with 10% FCS, non-essential amino acids, sodium pyruvate, penicillin and streptomycin in presence of 0.4 mg/ml G418 sulfate (all from Gibco, Invitrogen). The TC-1 cells were harvested with trypsin/EDTA (Gibco, Invitrogen), washed once with Hanks' balanced salt solution (HBSS, Gibco, Invitrogen) and resuspended into HBSS at a concentration of 2×10⁵ cells/ml. One hundred μl of the cell suspension were injected subcutaneously into the flank of mice.

### 6. Statistical Analysis

Statistical analysis were performed using Prism 4.00 for Windows (GraphPad software, San Diego, Calif.) by One-way ANOVA followed by Dunnet post test or by Student t test as indicated in the text or figure legends.

### 7. Chemical Synthesis

Sequences encoding E7 or E6 were synthesized, in whole or in part, using chemical methods well known in the art. Alternatively, E7 or E6 itself was produced using chemical methods to synthesize its amino acid sequence, such as by direct peptide synthesis using solid-phase techniques. Protein synthesis can either be performed using manual techniques or by automation. Automated synthesis is achieved, for example, using Applied Biosystems 431A Peptide Synthesizer (Perkin Elmer). Optionally, fragments of E7 or E6 were separately synthesized and combined using chemical methods to produce a full-length molecule.

The newly synthesized peptide is substantially purified by preparative high performance liquid chromatography. The composition of a synthetic E7 or E6 was confirmed by amino acid analysis or sequencing. Additionally, any portion of the amino acid sequence of E7 or E6 can be altered during direct synthesis and/or combined using chemical methods with sequences from other proteins to produce a variant polypeptide or a fusion protein.

E7 polypeptide synthesis was performed using automated Applied Biosystems 431A and 433A peptide synthesizers using FNOC-chemistry. The polypeptide contains an acidic C terminal and a free NH2-terminal.

### Results

E7-Specific CD8 T Cell Responses in PBMC of Mice Vaccinated sc or i.n. with E7₁₋₉₈ Administered with Different Adjuvants.

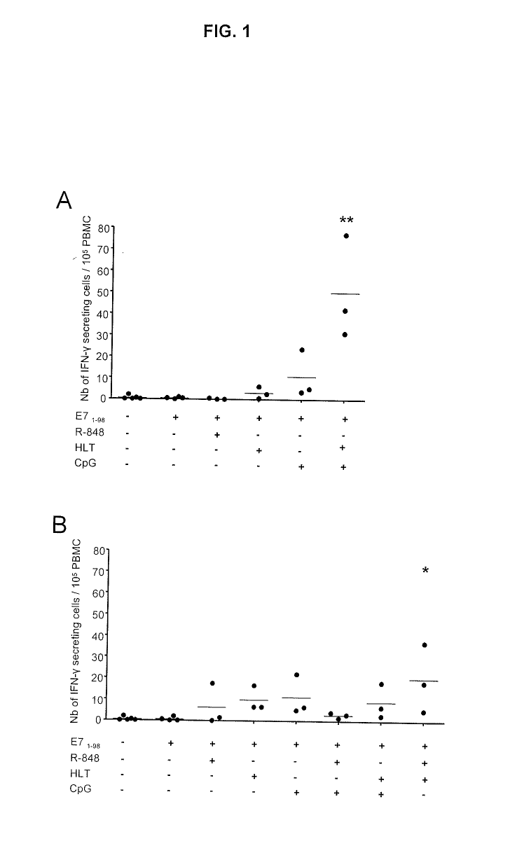

Two different Toll like receptor agonists, R-848 (a TRL-7 ligand) and CpG (a TLR-9 ligand), as well as a mutant form of a bacterial heat-labile enterotoxin (HLT, (Glueck, 2001)) were tested in a pilot experiment for their ability to enhance the E7-specific CD8 T cell responses induced by a single 50 μg dose of E7₁₋₉₈ Groups of 3 to 5 female C57B1/6 mice received the different inoculum by the sc (FIG. 1A) or i.n. routes (FIG. 1B). The immune responses were determined 7 days after immunization in the PBMC by ex-vivo IFN-γ ELISPOT using a well characterized H-2D^{b} restricted E7₄₉₋₅₇ peptide as detailed in the Material and Method section. A 50 μg sc or i.n. dose of E7₁₋₉₈ alone did not induce detectable E7-specific CD8 T cell responses, but addition of HLT or CpG induced significant responses in most mice, while R-848 did so in one out of three mice only after i.n. administration. These adjuvants were further tested in combinations of two and a significantly improved E7-specific CD8 T cell response was observed when combination of HLT and CpG were used for the sc route (p<0.01) and HLT and R-848 were used for the i.n. route (p<0.05). These two protocols were therefore used in the following experiment.

Amplitude and Kinetic of the E7-Specific CD8 T Cell Responses Induced in PBMC, Spleen, LN and CV Tissue

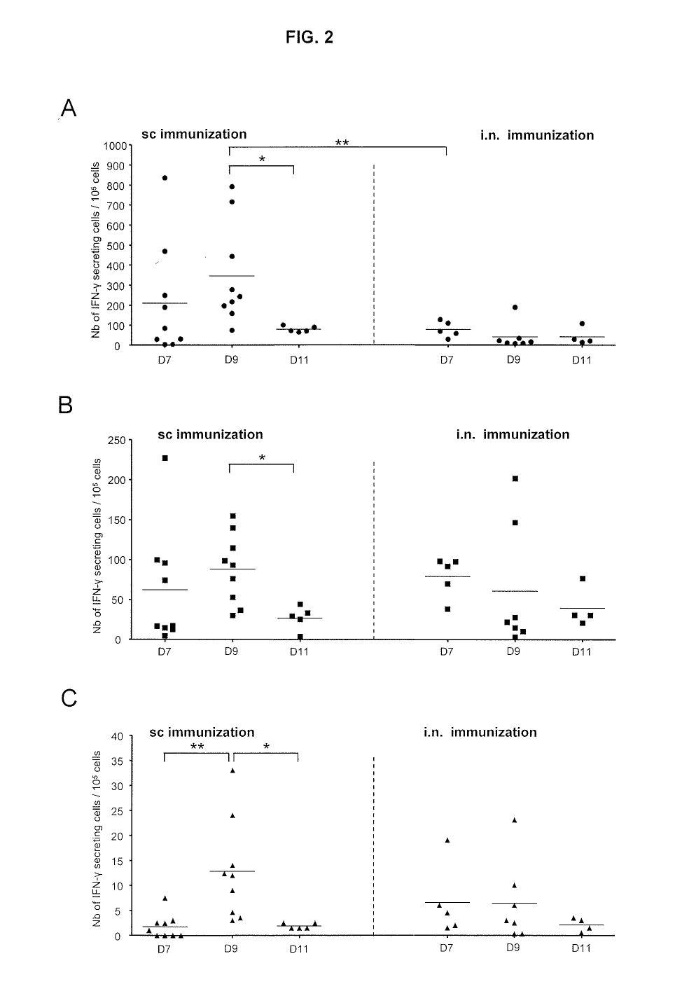

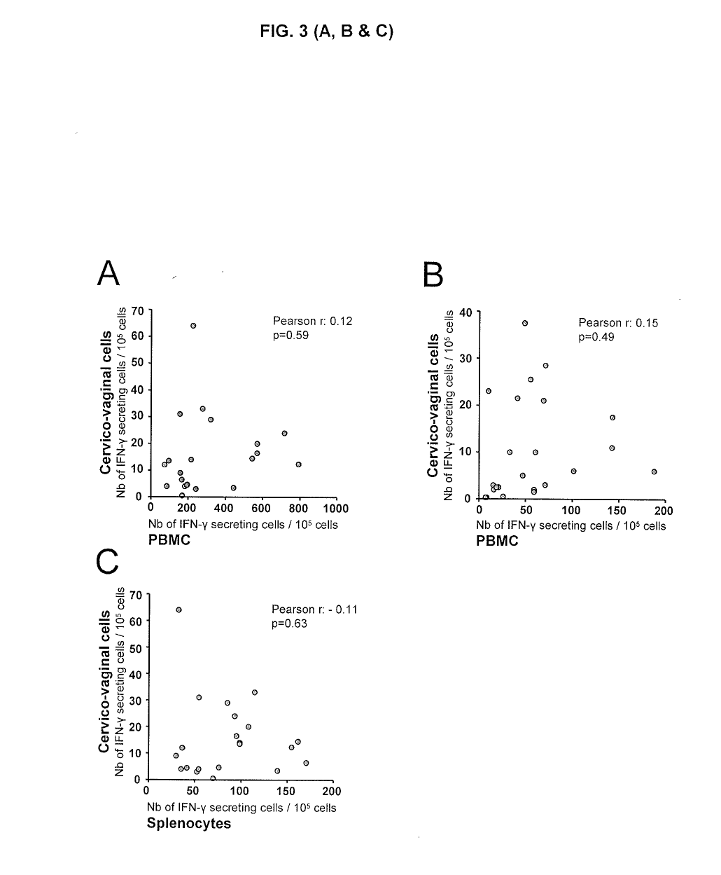

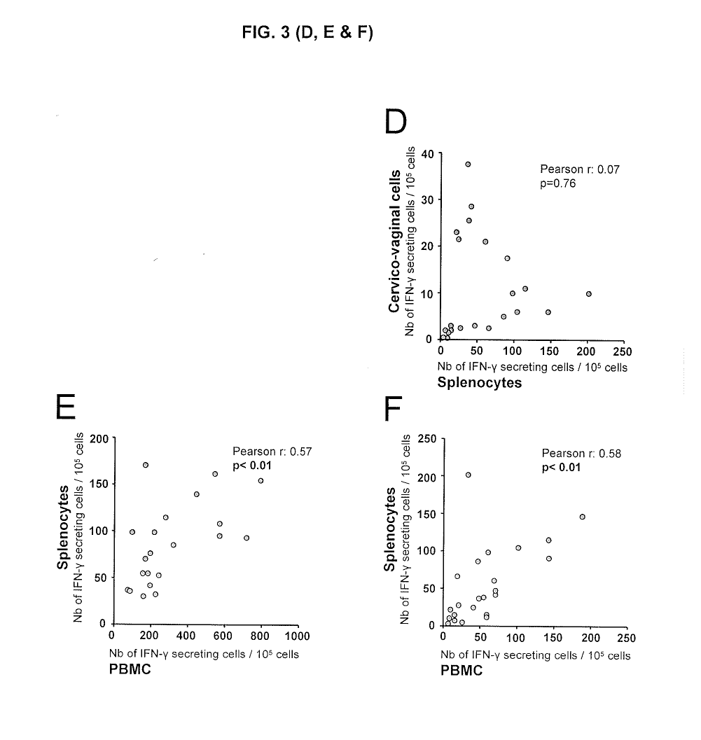

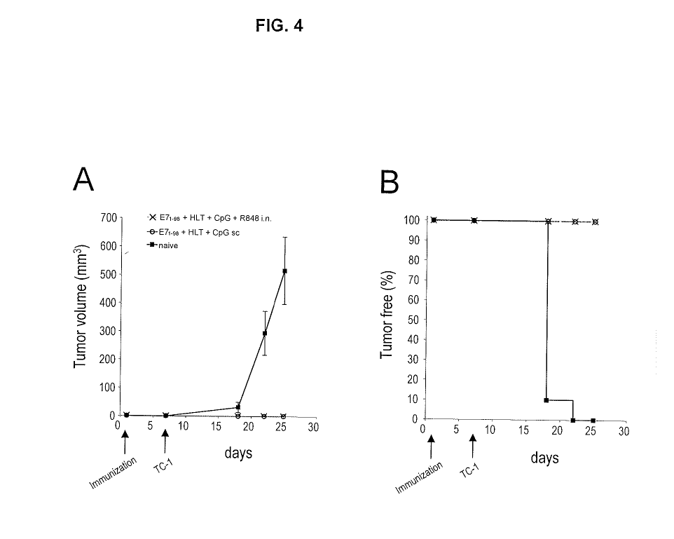

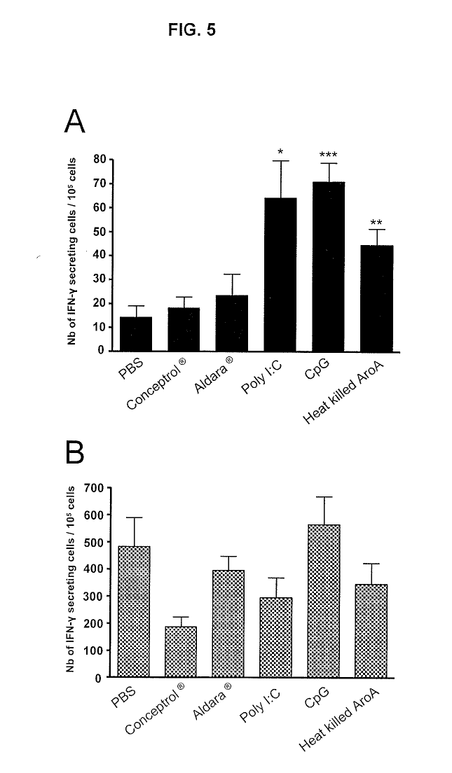

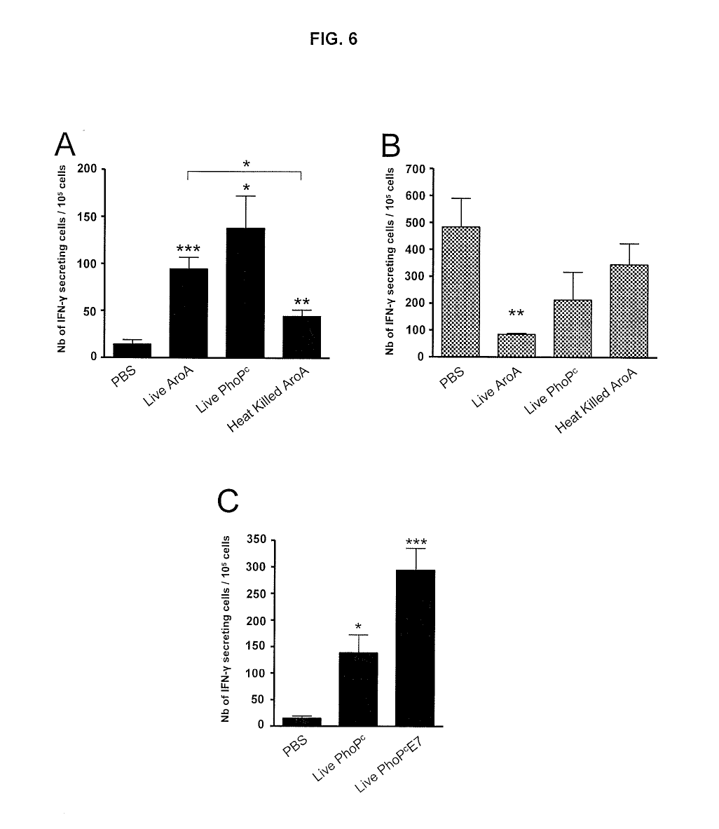

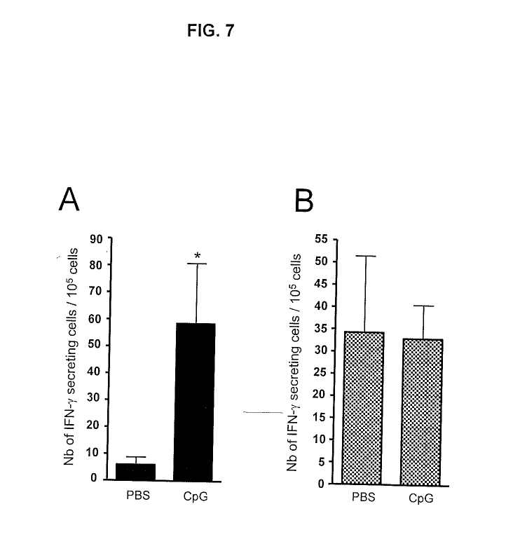

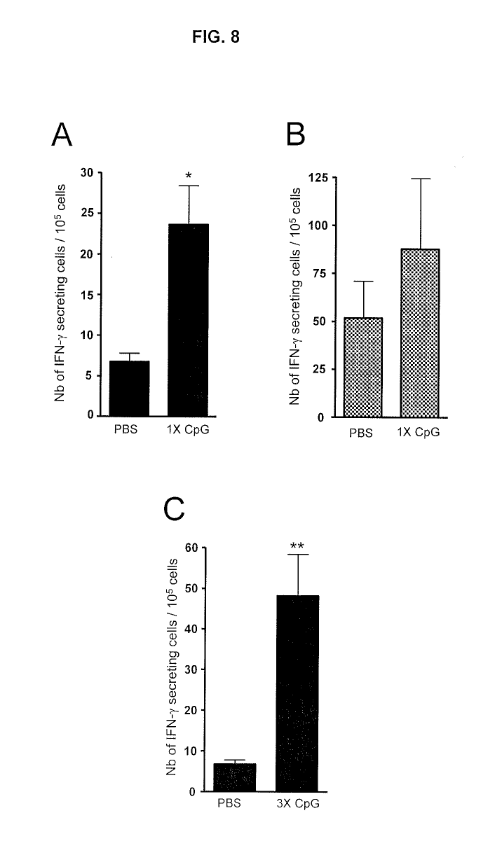

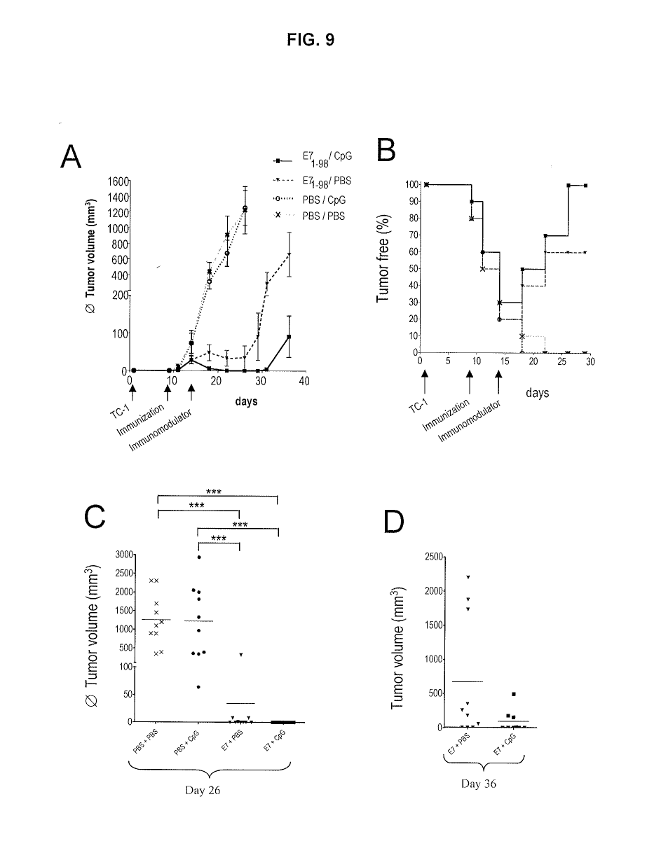

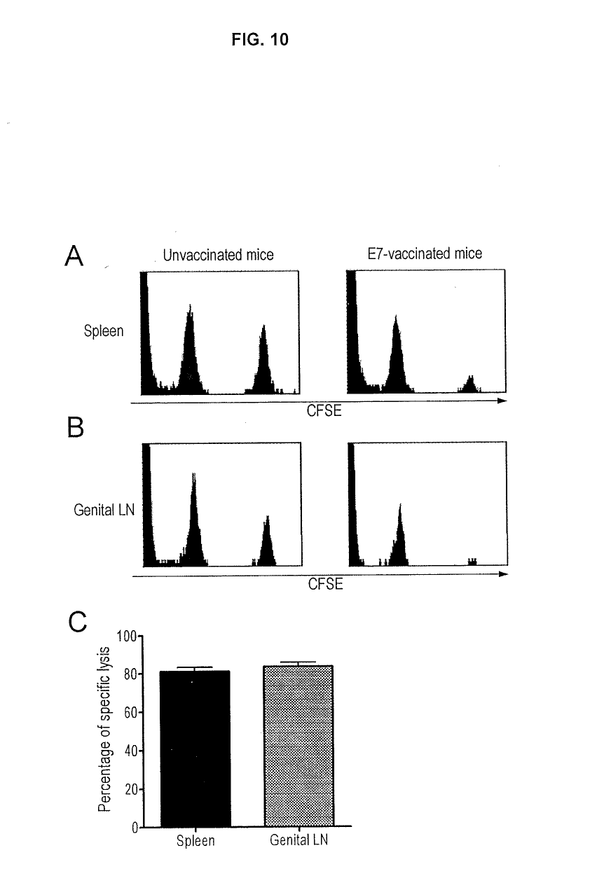

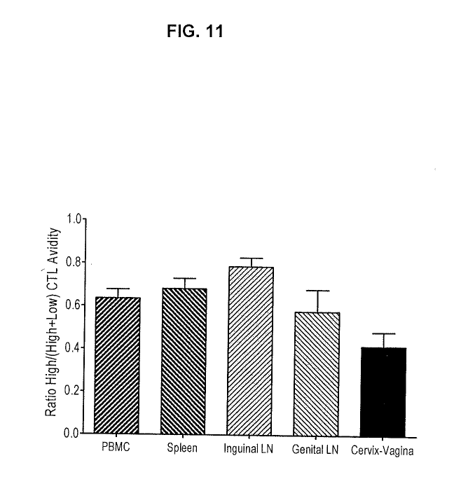

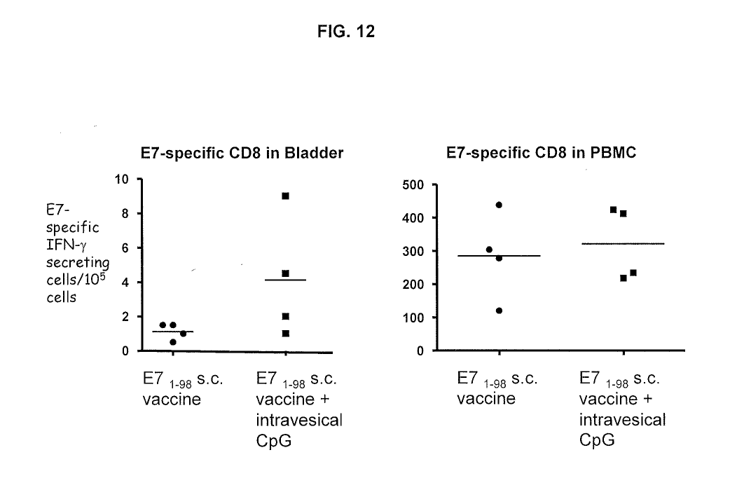

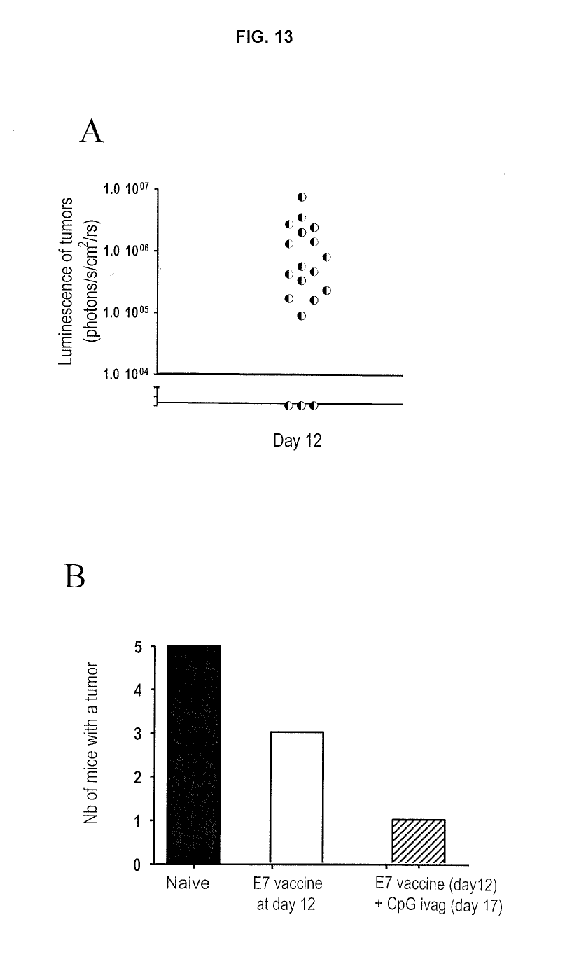

The kinetics of the CD8 T cell response were shown to peak in the periphery ca. 7-8 days after sc immunization with synthetic peptides (Miconnet et al., 2002) or VLP (Revaz et al., 2008) and maximal responses in CV tissues are expected to closely follow this pattern. However, this may differ with the routes of immunization or the use of adjuvants and thus may influence the comparisons of vaccination protocols thereafter. We have therefore precisely characterized the amplitude of the E7-specific CD8 T cell responses at day 7, 9 and 11 after sc and i.n. vaccination in PBMC, spleen and CV tissues (FIG. 2), as well as in draining LN (Table 1). Groups of 5 to 9 mice were immunized with 50 ng E7₁₋₉₈+CpG and HLT or +CpG and R-848 by sc or i.n. routes, respectively. Mice were sacrificed at day 7, 9 or 11 and PBMC, spleen, CV tissue, iliac LN (ILN, draining the sc site of immunization and CV tissue), inguinal LN (IGN, draining the sc site of immunization) and the cervical LN (CLN, draining the i.n. site of immunization) were harvested, purified and cell samples analyzed by ex-vivo IFN-γ ELISPOT. After sc immunization (FIG. 2, left side and Table 1) the highest E7-specific CD8 T cell responses were measured at day 9 after immunization in PBMC, spleen and CV tissue (FIGS. 2 A, B and C, left side), as well as in the draining LN (IGN and ILN, see Table 1). The responses were similar at day 7 except in CV tissues that exhibited a significantly lower response (mean E7-specific IFN-γ secreting cells±SEM of 1.8±0.8 as compared to 12.8±3.3 at day 9, p<0.01, see FIG. 1C.). At day 11, the responses were significantly lower than at day 9 in all these organs (p<0.05 for PBMC, spleen, CV and IGN or p<0.01 for ILN). After in immunization (FIG. 2 right side and Table 1), though the highest E7-specific CD8 T cell responses were measured at day 7 in the PBMC, spleen, ILN and CLN, the responses were similar at day 9 in CV tissues, non significantly decreased in PBMC and spleen, and only significantly decreased in the draining CLN, (p<0.01, see Table 1). At day 11, the responses were lower in these organs though only significantly different from day 7 in CLN (p<0.05). This suggests different kinetics of the CD8 T cell response after sc or i.n. immunizations, the latter appearing slower, though comparisons performed between day 9 for sc immunization and day 7 and/or 9 for i.n. immunization seem appropriate.

Comparison between the two routes of immunization showed significantly higher responses after sc immunization in PBMC and ILN (mean E7-specific IFN-γ secreting cells±SEM of 346.9±84.1 and 31.9±4.1, at day 9; as compared to 77.9±17.7 and 6.7±2.8, at day 7 of i.n. immunization for PBMC and ILN, respectively, p<0.01, see FIG. 2A and Table 1), while the responses were higher in the CLN after i.n. immunization (p<0.01, see Table 1) confirming the drainage of the nasal area by this LN. In contrast, the situation of ILN is less clear as these LN drain both the CV tissue and the skin near the base of the tail (the site of sc immunization). Indeed, when sc immunization of mice was performed in the skin in the neck area, the E7-specific CD8 T cell immune responses were similar in all organs (mean±SEM of 225.8±52.4, 86.8±19.2 and 17.1±4.5 as compared to 346±84.1, 88.3±14.6, and 12.8±3.3 for PBMC, spleen and CV, respectively), except in the IGN and ILN where they were significantly lower (mean±SEM of 9.9±4.5 and 10.6±3.5, as compared to 31.9±4.1 and 49.3±6.9, respectively, after sc at the base of the tail area at day 9, p<0.01), in agreement with the fact that they do not drain the skin of the neck area. In this case the E7-specific CD8 T cells responses measured in the ILN is similar to those measured after i.n. immunization (6.7±2.8 at day 7). Interestingly, sc immunization in the neck area also induced a significantly 5 fold higher E7-specific CD8 T cell response in PBMC than i.n. immunization (see Table 2, mean±SEM of 225.8±52.4 as compared to 40.4±24.8, p<0.001).Despite these differences in the peripheral response, the E7-specific CD8 T cell responses in CV tissues were only slightly but non significantly lower after i.n. immunization (see FIGS. 2 B and C).

Optimization of E7-specific CD8 T cell responses in CV tissues

In an attempt to optimize the E7-specific CD8 T cell responses in CV tissues different protocols of immunization were tested in groups of 4 to 9 mice (see Table 2). Sc immunization with a higher 100 μg dose of E7₁₋₉₈ or a double (week 0 and week 5) immunization with 50 μg E7₁₋₉₈ slightly augmented the E7-specific CD8 T cell responses particularly in the CV tissues, though not significantly In immunization with a third adjuvant, CpG, in addition to HLT and R-848, did not improve the specific immune response. Two (week 0 and week 5) in immunizations with 50 μg E7₁₋₉₈+the three adjuvants, as well as administration of 150 μg E7₁₋₉₈ delivered as a single dose or as three daily 50 μg doses, all improved the immune response in CV tissues, though not significantly (p=0.064 for 3×50 μg E7₁₋₉₈ i.n., as compared to 1×50 μg E7₁₋₉₈ i.n see Table 2). Finally, both optimized sc. or in immunization protocols were able to induce similarly high E7-specific CD8 T cell responses in CV tissue (ca.18 E7-specific IFN-γ secreting CD8 T cells/10⁵ cells). Interestingly however, the E7-specific CD8 T cell responses induced in PBMC remained 4-5 fold higher after sc immunization, which may suggest that homing to the CV tissues is not favored by this route.

Individual E7-Specific CD8 T Cell Responses Measured in CV Tissues do not Correlate to the Responses Measured in PBMC or Spleen.

Because of the difficulty to analyze the CV tissues especially when dealing with small biopsies in clinical trials, we have further examined how the specific CD8 T cell responses measured in blood could provide valuable information upon the responses measured in CV tissue. For this purpose we have plotted the E7-specific CD8 T cell responses measured in CV of individual mice against the responses measured in their PBMC (see FIGS. 3 A and B, data pooled from Table 2). Our data show that neither for sc or in immunization there is a correlation between CD8 T cell responses in PBMC and CV tissues (Pearson r=0.12 and 0.15 for sc and in immunization, respectively). In pre-clinical testing with mice, the immune responses are often measured in the spleen, however our data again did not show correlation with the immune responses measured in CV tissue (see FIGS. 3 C and D). As expected, a correlation was however found when the E7-specific CD8 T cell responses measured in PBMC were plotted against the responses measured in the spleen of the same animals (Pearson r=0.57 and 0.58, p<0.01 for sc and in immunization, respectively).

Both sc and i.n. immunization with the E7₁₋₉₈ vaccine provided anti-tumor protection. In the absence of mice bearing genital HPV-tumors, we tested the therapeutic effect of the sc and i.n. E7₁₋₉₈ vaccines by using the TC-1 tumor protection assay. Groups of 9 mice were sc or i.n. vaccinated with E7₁₋₉₈+HLT and CpG or +HLT, CpG and R848, respectively. One week after vaccination, the mice were challenged with 2×10⁴ TC-1 tumor cells and monitored twice a week for tumor growth. The tumors only grew in the unvaccinated mice while all mice vaccinated i.n. or s.c. remained tumor free for at least 25 days (see FIGS. 4 A and B). This showed that a single i.n. or sc vaccination with E7 1-98+adjuvants was able to prevent E7-tumor growth.

### E7-Specific Cytotoxic T Cells are Induced In Vivo in Vaccinated Mice

To determine whether tumor regression was associated with the presence of E7-specific CD8 CTL, Applicants used an in vivo cytotoxicity assay. This method involves the in vivo selective lysis of fluorescently labeled E7₄₉₋₅₇-pulsed splenocytes (CFSE-high) that have been i.v. transferred 7 days after vaccination. Our data shows 15 hours after i.v. transfer a high specific lysis as compared to unpulsed CFSE-low labeled splenocytes in both spleen and genital draining LN (see FIGS. 10 A and B, for a representative experiment), reaching a mean percentage of specific lysis of 80% in both organs (see FIG. 10 C). The presence of E7-specific CTL in the LN draining the genital tract suggests that those CTL are also present within the CV mucosa. (indeed E7-specific CD8 T cells in the CV mucosa are detected by INF-γ ELISPOT).

### High Avidity E7-Specific IFN-γ Secreting CD8 T Cells are Generated in All Organs Examined Including the CV Mucosa.

Applicants further examined the avidity of the E7-specific CD8 T cells responses induced. For this purpose PBMC, spleen, inguinal and genital LN as well as CV were harvested from a group of 5 mice that had received the E7 vaccine 8 days earlier and were assayed by IFN-γ ELISPOT using 2 log dilutions of the E7₄₉₋₅₇ peptide. E7-specific CD8 T cells exhibiting a high-avidity, i.e. T cells activated with 10⁻¹⁰ peptide, were detected in all organs examined In order to quantify and compare the level of high-avidity effector CD8 T cells generated in the different organs, the ratio between high-avidity/high-avidity+low-avidity, i.e. T cells activated with 10⁻⁶ M peptides, were calculated and shown in FIG. 11. High-avidity E7-specific CD8 T cells represented between 40 and 80% of the E7-specific CD8 T cells measured in the different organs. Interestingly the percentage of high-avidity CTL in CV was only slightly lower than the percentage found in the other organs (only significantly lower when compared to inguinal LN), thus suggesting that a single s.c. E7 vaccine dose was sufficient to generate mucosal high-avidity CTL.

### Example 2

### Application of Immunostimulatory Molecules on the Vaginal Mucosa of Mice after sc or i.n. Immunization with E7₁₋₉₈ Strongly Enhance the E7-Specific CD8 T Cell Response in the Cervico-Vaginal Tissue

We have shown that both sc and in immunization with a synthetic polypeptide vaccine, E7₁₋₉₈, administered with specific adjuvants, can induce high E7-specific CD8 T cell responses in the cervico-vaginal (CV) tissue of vaccinated mice.

However, the immunosuppressive microenvironment of the cervical mucosa favors the development of HSIL and/or cervical cancer, which correlates with local type II cytokines, absence of IFN-γ, reduced density and functions of Langerhans cells, as well as increased proportion of regulatory T cells in the draining lymph nodes of cervical cancer. In addition, there is little or no inflammation at the site of primary HPV infection and HPV may be capable to suppress the host immune response (Kleine-Lowinski et al., 2003).
Therefore, here we show that to enhance therapeutic properties an additional value to HPV vaccination may be to change the local microenvironment within the mucosa. The aims would be (i) to attract and/or activate non specifically in the cervico-vaginal mucosa additional vaccine-specific T cells that have been induced by a prime vaccination (ii) to induce a micro-environment of inflammatory cytokines and chemokines that may relieve the local immunological suppressive status and that act on tumor-stroma interactions in order to promote tumor regression.

### Results

Effect of Immunomudolatory Agents Applied on the Vaginal Mucosa of Mice after sc Vaccination with the Synthetic Vaccine E7₁₋₉₈+CpG and HLT

In order to attract and/or activate in the CV tissue E7-specific CD8 T cells, it is necessary to first, immunize mice with an E7-vaccine and to second, apply locally in the vagina an immunomudulatory molecule/agent at a time when the vaccine E7-specific T cells circulate/peak in the blood. For this purpose groups of four C57B1/6 mice were sc immunized with E7₁₋₉₈+CpG and HLT at day 1, 5 days later the following molecules were administered intravaginally (ivag): PBS (control), Conceptrol® (a cream containing nonoxynol-9, a spermicide and disrupter of the cervico-vaginal epithelium (Roberts et al., 2007), Aldara® (a cream containing imiquimod, an imidazoquinolines molecules, TLR7 and δ agonist that is used to treat anogenital warts, poly I: poly C (pI:C, polyriboinosinic:polyribocytidylic acid, a TLR-3 agonist), CpG (a TLR-9 ligand), or heat-killed bacteria (Salmonella typhimurium attenuated AroA strain, as TLR 2, 4 and 5 ligands). Prior to ivag treatments, mice were synchronized in a diestrus-like state by sc injection with 0.1 μg β-estradiol and 24 h later with 2.5 mg DepoProvera, this is to avoid variation in the ivag immunomodulatory effects along the menstrual cycle of mice. The mice were sacrificed three days after ivag treatments and the E7-specific CD8 T cell responses were examined by ex-vivo IFN-γ ELISPOT using a well characterized H-2 Db restricted E7₄₉₋₅₇ peptide in CV tissue (See FIG. 5 A), PBMC (see FIG. 5 B) and in spleen, inguinal LN (IGN), iliac LN (ILN) and cervical LN (CLN). Ivag pI:C, CpG and heat-killed AroA, all strongly enhanced the E7-specific CD8 T cell responses in CV tissue as compared to PBS (p<0.05 and p<0.01, respectively). This enhancement was restricted to the CV tissue as the E7-specific CD8 T cell responses were similar to the group treated with ivag PBS in PBMC (see FIG. 5 B) as well as in spleen and the other LN examined (data not shown) suggesting that the effects of ivag immunomodulators were also restricted to the site of application, at least at this early time point. In this experiment the treatments with Aldara and Conceptrol only slightly enhanced the E7 response in the CV tissues, experiments using a more potent imidazoquinolines molecule (Resiquimod) and nonoxynol-9 in liquid formulation are ongoing.

Effect of Live Attenuated Salmonella Applied on the Vaginal Mucosa of Mice after sc Vaccination with the Synthetic Vaccine E7₁₋₉₈+CpG and HLT

Our data show that ivag heat killed AroA were able to enhance the specific responses in the CV tissue. Because recombinant live attenuated Salmonella are known to be immunogenic and to induce transient inflammatory reaction by the ivag route, we further investigated here their effect on the vaccine specific CD8 T cell response. Two groups of mice that were sc immunized with E7₁₋₉₈+CpG and HLT at day1, received live recombinant AroA or PhoP^{e} bacteria at day 6. Mice were sacrificed three days later and E7-specific CD8 T cell responses examined in CV tissue (FIG. 6 A), PBMC (FIG. 6 B), spleen, IGN, ILN and CLN. Both attenuated live bacteria induced a ca. 10-fold significant enhancement of the vaccine-specific immune response in CV tissue as compared to PBS. The E7-response induced in CV by the live AroA was also significantly higher than that induced by the heat killed bacteria (p<0.05). Interestingly, ivag live bacteria appeared to reduce the E7-specific CD8 T cell response in PBMC (statistically significant for live AroA, p<0.01) and in spleen (data not shown), may be reflecting depletion of E7-specific CD8 T cells from the periphery upon accumulation in the CV tissue. The use of live attenuated Salmonella that express the E7 oncogene may be particularly interesting has the effect of ivag immunomodulator may be combined to ivag delivery of a booster vaccination (E7). Indeed higher E7-specific CD8 T cell responses were observed in CV tissue when a PhoP^{e} Salmonella expressing E7 was used as ivag immunomodulator (see FIG. 6 C).

Effect of CpG Applied on the Vaginal Mucosa of Mice after sc or i.n. Vaccination with the E7 Vaccine.

Effect of ivag immunomodulators upon i.n. immunization was examined using CpG. Two groups of mice that received the i.n. E7 vaccine at day 1, received ivag PBS or ivag CpG at day6 and mice were sacrificed three days later. The E7-specific CD8 T cell response showed a 10-fold increase in CV tissue as compared to PBS though not significantly (p=0.058, see FIG. 7 A), while no differences were observed in PBMC (FIG. 7 B) and the other organ examined (data not shown). Interestingly, the increase of the vaccine-specific CD 8 T cell response in CV tissue upon ivag CpG after i.n. immunization was greater (10-fold) than after sc immunization (4-fold). This is despite of the fact that the vaccine-specific CD8 T cell response was 10-fold higher in the PBMC of the sc immunized mice (mean E7-specific CD8 T cells/10⁵ of ca. 500) as compared to i.n. immunized mice (mean of ca 30).This observation may suggest a greater effect of ivag immunomodulator upon mucosal vaccination.

The duration of ivag CpG was also evaluated after sc immunization with the E7-vaccine. Two groups of mice that received the sc E7 vaccine at day1, received ivag PBS or ivag CpG at day 6 and mice were sacrificed at day 15. The E7-specific CD8 T cell responses in CV tissues after ivag CpG were significantly higher than after ivag PBS (FIG. 8 A), while the vaccine-specific responses in PBMC (FIG. 8 B) and all organs examined were similar. This show that the effect of ivag CpG at day 6 is still visible 9 days later, at a time when the CD8 T cell response in CV is already greatly decreased (mean E7-specific CD8 T cells/10⁵ cells of ca 5 as compared to 14 at day 9). At this later time point (day 15) however, ivag CpG appears to have an additional non E7-specific effect restricted to the CV tissue as the background of IFN-γ secreting cells was higher in those samples (mean of IFN-γ secreting cells/10⁵ of ca 10 as compared to 1.5 at day 9).

Applicants further evaluated the effect of three consecutive ivag CpG treatments on the E7-specific CD8T cell response. After sc vaccination with the E7 vaccine, ivag CpG were administered at day 6, 9 and 12, while control mice received ivag PBS at the same time points. The mice were sacrificed at day 15 and the E7-specific IFN-γ secreting cells examined in all tissues. The E7-specific CD8 T cell responses was strongly increased in CV tissue after 3 ivag CpG treatments as compared to the control mice (see FIG. 8 C) though similar to the E7-specific response measured in CV tissues at day 9 after a single ivag CpG treatment. This suggests that consecutive ivag applications of immunonostimulants may maintain a very high E7-specific CD8 T cell response in the CV tissue. However, this triple ivag CpG treatment also induced some non E7-specific IFN-γ secreting cells in the CV tissue, the draining ILN and in PBMC. Indeed the background number of IFN-γ secreting cells was increased in these organs as compared to the PBS treated mice (mean IFN-γ secreting cell/10⁵ cells of 50, 75 and 78 in CV, ILN and PBMC, respectively, as compared to 1, 1 and 5 in PBS treated mice or to 10, 2 and 4 in mice that received a single ivag CpG treatment).

Anti-Tumor Effect of a Topical Immunomodulatory Treatment after sc Immunization with the E7 Vaccine.

In the absence of mice bearing HPV genital tumor to assess the anti-tumor potential of our E7 vaccine alone or combined with a topical immunomodulatory treatment we have used HPV tumor cells (TC-1,) that can be sc injected in the flank of syngeneic mice. We have set up a tumor protection assay in a therapeutic setting where the tumor cells are first injected, then the E7 vaccine is administered and finally CpG are sc injected in the skin next to the tumors. For this purpose 4 groups of 10 C57BL/6 mice received s.c. in their right flank 2×10⁴ TC-1 tumor cells, 8 days later when half of the mice had palpable tumors, two groups of 10 mice received sc at the base of the tail 50 μg E7₁₋₉₈+HLT and CpG, while the two other groups received PBS, 5 days later (at day 14) one group of E7-vaccinated mice and one group of PBS-vaccinated mice received 30 μg of CpG sc next to their tumor, while the two others groups received PBS. The volumes of the tumore were recorded twice a week, if tumor volumes were >2500 m3 the mice had to be sacrificed for ethical reason. Mean tumor volumes are shown in FIG. 9 A. The last time point at which all mice were still alive was day 26 and individual tumor volumes are shown at this time point in FIG. 9 C. The mice vaccinated with PBS rapidly developed growing tumors, irrespective of the topical treatment with CpG, while the E7 vaccinated mice had very slow growing tumors. Upon sc CpG treatment the E7-vaccinated mice completely regressed their tumors and become tumor free at day 26 (see FIG. 9 B). At this time point 40% of the E7-vaccinated mice harbored a small tumor. Our data thus demonstrate a high efficacy our E7 vaccine in this therapeutic setting (compare to PBS vaccinated mice), as well as the efficacy of the topical CpG treatment that allow a complete though transient regression of the tumors in all mice.

The fact the CpG treatment was totally inefficient in un-vaccinated mice at this dose and time point demonstrate that its effect is mediated by E7-specific CD8 T cells that were induced by the E7-vaccine and not some unspecific anti-tumor event. The mechanism of action of topical immunomodulatory treatment following a prime vaccination is thus different, though probably related, from other anti-tumor activity of TLR agonists as previously reported.

TABLE 1

| | sc immunization | | | i.n. immunization | | |
|---|---|---|---|---|---|---|
| organs | Day 7 | Day 9 | Day 11 | Day 7 | Day 9 | Day 11 |
| CL | 6.4 ± 2.7 | 11.2 ± 2.9^{†} | 6.1 ± 1.6 | 30.2 ± 6.1^{a} | 7.8 ± 1.8 | 8.6 ± 4.3 |
| | N = 9 | N = 8 | N = 5 | N = 5 | N = 7 | N = 4 |
| IL | 35.6 ± 10.7 | 31.9 ± 4.1^{b} | 11.8 ± 1.7 | 6.7 ± 2.8^{‡} | 3.5 ± 0.9 | 5.1 ± 1.8 |
| | N = 9 | N = 9 | N = 5 | N = 5 | N = 7 | N = 4 |
| IG | 42.8 ± 18.0 | 49.3 ± 6.9^{c} | 22.4 ± 5.2 | n. a | n.a | n.a |
| | N = 9 | N = 9 | N = 5 | | | |

^{a}statistically higher than day 9 (p < 0.01) and day 11 (p < 0.05) i.n. immunization
^{b}statistically higher than day 11 sc immunization (p < 0.01)
^{c}statistically higher than day 11 sc immunization (p < 0.05)
^{†}statistically lower than day 7 after i.n. immunization p < 0.01
^{‡}statistically lower than day 9 after sc immunization p < 0.01

TABLE 2

| | i.n. immunization (E7₁₋₉₈ + HLT + R848) | | | | | | | |
|---|---|---|---|---|---|---|---|---|
| | | | | E7₁₋₉₈ 50 μg + | E7₁₋₉₈ 50 μg + | sc immunization (E7₁₋₉₈ + CpG + HLT) | | |
| | | E7₁₋₉₈ 50 μg + | E7₁₋₉₈ 150 μg + | CpG | CpG | | | E7₁₋₉₈ 50 μg |
| | E7₁₋₉₈ 50 μg | CpG | CpG | D1, D2, D3 | D1 and W5 | E7₁₋₉₈ 50 μg | E7_{1 98} 100 μg | D1 and W5 |
| PBMC | 40.4 ± 24.8^{a} | 63.3 ± 14.5 | 67.8 ± 26.6 | 63.0 ± 7.3 | 68.8 ± 18.2 | 346.9 ± 84.1 | 370.3 ± 116.0 | 433.1 ± 133.9 |
| | N = 7 | N = 8 | N = 4 | N = 4 | N = 5 | N = 9 | N = 4 | N = 5 |
| Spleen | 60.6 ± 30.0 | 47.7 ± 16.5 | 72.8 ± 13.9 | 43.4 ± 7.5 | 40.7 ± 8.8 | 88.3 ± 14.6 | 78.0 ± 13.8 | 217.4 ± 93.3 |
| | N = 7 | N = 8 | N = 4 | N = 4 | N = 5 | N = 9 | N = 4 | N = 5 |
| Cervix-vagina | 6.4 ± 3.0 | 6.7 ± 2.9 | 16.9 ± 7.5 | 18.5 ± 5.4 | 11.5 ± 1.6 | 12.8 ± 3.3 | 17.9 ± 5.6 | 17.9 ± 5.4 |
| | N = 7 | N = 8 | N = 4 | N = 4 | N = 5 | N = 9 | N = 4 | N = 5 |

^{a}mean ± SEM E7-specific CD8 T cells/10⁵ cells

### Example 3

Anti-Tumor Effect of s.c. Vaccination Followed by a Intravaginal Immunostimulant

A new murine model that harbors cervico-vaginal HPV16 tumor is necessary in order to assess the effect of intravaginal immunostimulants. An interesting approach for inducing local tumors is to establish orthotopic murine models, where tumor cells are implanted at the site of the original tumors. In the most similar situation to cervical cancer i.e. bladder cancer, this is achieved through intravesical instillation of tumor cells after a chemical or mechanical wounding of the bladder epithelium. Toward the establishment of an orthotopic murine model for cervical cancer, we have thus induced tumor take after ivag instillation of TC-1 cells following nonoxynol-9 treatment. Indeed, the spermicid/detergent nonoxynol-9 was recently shown to disrupt the epithelium so that access of HPV pseudovirions to the basal layer of the epithelium and infection can occur. Here we have exploited this wounding of the epithelium for introducing TC-1 cells that can be trapped during healing. In addition, we have engineered TC-1 cells that express the luciferase gene (TC-1-luc) so that the intravaginal tumors can be followed by in vivo bioluminescence imaging after i.p. injection of luciferine with a Xenogen camera. Our data demonstrated 90% tumor take 12 days after ivag instillation of 12′500 TC-1 luc cells (see FIG. 13 A). Three groups of 5 mice harboring ivag TC-1-luc tumors were compared: two groups of mice received 12 days after ivag TC1-luc cells one dose of the sc E7₁₋₉₈+CpG+HLT vaccine either alone or followed by an ivag instillation of 100 μg CpG, while the third group was left unvaccinated (see FIG. 13 B). Our data show that 5/5 naïve mice developed a tumor, while 68 days after TC-1-luc challenge only 1/5 mice that received both the vaccine and the ivag CpG exhibited a tumor, while 3/5 mice developed a tumor if they received the vaccine alone. These data thus suggest that vaccination followed by ivag CpG is more efficient to diminish the tumors than vaccination alone.

### Example 4

### Effect of Intravesical Instillation of Immunostimulant (CpG) on the E7-Specific CD8 T Cell Response in the Bladder

Applicants have tested whether application of immunostimulant into another topical site i.e. intravesical can augment the vaccine-specific response in this mucosal site i.e. the bladder. For this purpose mice received the sc E7 vaccines (as a model vaccine) followed 5 days later by a 100 ug dose of CpG instillated intravesically through a catheter inserted into the urethra. Our preliminary data shows trend towards a 4-fold increase in the E7-specific CD8 T cells measured in the bladder mucosa after intravesical application of CpG (see FIG. 12).

### REFERENCES

Balmelli, C., Demotz, S., Acha-Orbea, H., De Grandi, P., and Nardelli-Haefliger, D. (2002). Trachea, lung, and tracheobronchial lymph nodes are the major sites where antigen-presenting cells are detected after nasal vaccination of mice with human papillomavirus type 16 virus-like particles. J Virol 76, 12596-12602.
Freytag, L.C., and Clements, J. D. (2005). Mucosal adjuvants. Vaccine 23, 1804-1813.
Glueck, R. (2001). Pre-clinical and clinical investigation of the safety of a novel adjuvant for intranasal immunization. Vaccine 20, S42-S44.
Kleine-Lowinski, K., Rheinwald, J. G., Fichorova, R. N., Anderson, D. J., Basile, J., Münger, K., Daly, C. M., Rösl, F., and Rollins, B. J. (2003). Selective suppression of monocyte chmoattractant protein-1 expression by human ppipillomavirus E6 and E7 oncoproteins in human cervical epithelial and epidermal cells. Int J Cancer 107, 407-415.
Lee et al. (2003) Differential modulation of Toll-like receptors by fatty acids: preferential inhibition by n-3 polyunsaturated fatty acids. Journal of Lipid Research 44: 479-486
Lowy, D., and Schiller, J. (2006). Prophylactic human papillomavirus vaccines. J Clin Invest 116, 1167-1173.
Miconnet, I., Koenig, S., Speiser, D., Krieg, A., Guillaume, P., Cerottini, J. C., and Romero, P. (2002). CpG are efficient adjuvants for specific CTL induction against tumor antigen-derivec peptide J Immunol 168, 121-1218.
Revaz, V., Debonneville, A., Bobst, M., and Nardelli-Haefliger, D. (2008). Monitoring of vaccine-specific IFN-g induction in the genital mucosa of mice by real-time RT-PCR. Clin Vacc Immunol, in press.
Stephenson I, Zambon M C, Rudin A, Colegate A, Podda A, Bugarini R, et al. (2006). Phase I evaluation of intranasal trivalent inactivated influenza vaccine with nontoxigenic Escherichia coli enterotoxin and novel biovector as mucosal adjuvants, using adult volunteers. J Virol; 80(10):4962-70
Stern P L Immune control of human papillomavirus (HPV) associated anogenital disease and potential for vaccination. J Clin Virol 2005; 32S: S72-S81.

1-32. (canceled)

33. A method for treating an infection or disease or lesion in a subject comprising
i) administering a vaccine comprising an antigen, and
ii) at a determined time which is the time just before there is a peak in the vaccine specific T cell response, subsequently applying one or several consecutive dose of an immunostimulant at a site where the infection, the lesion or a disease is present.

34. The method of claim 33, wherein the infection is selected from the group comprising Anthrax, Candida, Chlamidia, Diphtheria, Hepatitis A, Hepatitis B, Haemophilus influenzae type b (Hib), Human Papillomavirus (HPV), HIV, Influenza (Flu), Japanese encephalitis (JE), Lyme disease, Measles, Meningococcal, Monkeypox, Mumps Pertussis, Pneumococcal, Polio, Rabies, Rotavirus, Rubella, Shingles (Herpes Zoster), HSV, Smallpox, Tetanus, Typhoid, Tuberculosis (TB), Varicella (Chickenpox) and Yellow Fever.

35. The method of claim 33, wherein the antigen is a tumor antigen.

36. The method of claim 35, wherein the disease is selected from the cancer group comprising melanoma, colon cancer, bladder cancer, breast cancer, prostate cancer, lung cancer carcinoma, lymphoma, blastoma, sarcoma, liposarcoma, neuroendocrine tumor, mesothelioma, schwanoma, meningioma, adenocarcinoma, leukemia, lymphoid malignancy, squamous cell cancer, epithelial squamous cell cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, hepatoma, rectal cancer, colorectal cancer, endometrial or uterine carcinoma, salivary gland carcinoma, kidney or renal cancer, vulval cancer, thyroid cancer, hepatic carcinoma, anal carcinoma, penile carcinoma, testicular cancer, esophageal cancer, a tumor of the biliary tract, and head and neck cancer.

37. The method of claim 36, wherein the disease is bladder cancer and the immunostimulant is applied in the bladder.

38. The method of claim 33 for treating a Human Papillomavirus infection or a related disease or lesion in a subject comprising
i) administering a vaccine comprising a complete synthetic E6 and/or E7 polypeptide of an HPV type and an adjuvant, or any vaccine suitable for treating a Human Papillomavirus (HPV) and,
ii) at a determined time which is the time just before there is a peak in the vaccine specific T cell response, subsequently applying one or several consecutive dose of an immunostimulant at a site where the Human Papillomavirus infection, the related disease or the lesion is located.

39. The method of claim 38, wherein the Human Papillomavirus infection related disease is Cervical intraepithelial neoplasia (CIN) type I, II et III, Vulvar IN I, II III, Vulvar intraepithelial neoplasia (VIN) I,II II, Condyloma, Anal IN, I, II, III, genital warts, ano-genital cancer and non genital HPV-related lesions.

40. The method of claim 33, wherein the at least one immunostimulant of step ii) is a radiation or an agent able to recruit immune effectors cells and/or relieve local immunosuppressive status via the induction of local cytokines and chemokines.

41. The method of claim 40, wherein the agent able to recruit immune effectors cells and/or relieve local immunosuppressive status is selected from the group comprising TLR-agonists, pro-inflammatory molecules and live bacterial or viral vaccines strains.

42. The method of claim 41, wherein the toll-like receptor agonist is selected from the group comprising an agonist of TLR 2, TLR 3, TLR 4, TLR 5, TLR 7/8 or TLR 9.

43. The method of claim 42, wherein
i) the TLR 2 agonist is selected from the group comprising Pam₃CysSerLys₄ and macrophage-activating lipopeptide-2,
ii) the TLR 3 agonist is poly I:C, iii) the TLR 4 agonist is LPS, iv) the TLR 5 agonist is flagellin, v) the TLR 7/8 agonist is selected from the imidazoquinoline group comprising immiquimod and resiquimod, and vi) the TLR 9 agonist is CpG.

44. The method of claim 41, wherein the pro-inflammatory molecule is selected from the group comprising detergent, spermicide (nonoxynol-9), microbicide and cytokines.

45. The method of claim 41, wherein the live bacterial or viral vaccine strain is selected from the group comprising live attenuated Salmonelle enterica serovar Typhimuium and Typhi strain, Bacille Calmette Guérin (BCG) strains, live attenuated Listeria monocytogenes strains, Lactococcus lactis strain, Streptococcus gordonii strain, attenuated Candida strains, attenuated Chlamydia strains, Modified vaccinia Ankara (MVA) strains and Nyvac strains and FMSE (Tick-borne encephalitis virus vaccine)

46. The method of claim 38, wherein the site where the Human Papillomavirus infection, the related disease or the lesion is located is the genital mucosa.

47. The method of claim 33, wherein said one or several consecutive dose of an immunostimulant is applied topically.

48. The method of claim 34 for treating a Human Papillomavirus infection or a related disease or lesion in a subject comprising
i) administering a vaccine comprising a complete synthetic E6 and/or E7 polypeptide of an HPV type and an adjuvant, or any vaccine suitable for treating a Human Papillomavirus (HPV) and,
ii) at a determined time which is the time just before there is a peak in the vaccine specific T cell response, subsequently applying one or several consecutive dose of an immunostimulant at a site where the Human Papillomavirus infection, the related disease or the lesion is located.

49. The method of claim 34, wherein the at least one immunostimulant of step ii) is a radiation or an agent able to recruit immune effectors cells and/or relieve local immunosuppressive status via the induction of local cytokines and chemokines.

50. The method of claim 35, wherein the at least one immunostimulant of step ii) is a radiation or an agent able to recruit immune effectors cells and/or relieve local immunosuppressive status via the induction of local cytokines and chemokines.

51. The method of claim 36, wherein the at least one immunostimulant of step ii) is a radiation or an agent able to recruit immune effectors cells and/or relieve local immunosuppressive status via the induction of local cytokines and chemokines.

52. The method of claim 37, wherein the at least one immunostimulant of step ii) is a radiation or an agent able to recruit immune effectors cells and/or relieve local immunosuppressive status via the induction of local cytokines and chemokines.

53. The method of claim 38, wherein the at least one immunostimulant of step ii) is a radiation or an agent able to recruit immune effectors cells and/or relieve local immunosuppressive status via the induction of local cytokines and chemokines.

54. The method of claim 39, wherein the at least one immunostimulant of step ii) is a radiation or an agent able to recruit immune effectors cells and/or relieve local immunosuppressive status via the induction of local cytokines and chemokines.

55. The method of claim 34, wherein said one or several consecutive dose of an immunostimulant is applied topically.

56. The method of claim 35, wherein said one or several consecutive dose of an immunostimulant is applied topically.

57. The method of claim 36, wherein said one or several consecutive dose of an immunostimulant is applied topically.

58. The method of claim 37, wherein said one or several consecutive dose of an immunostimulant is applied topically.

59. The method of claim 38, wherein said one or several consecutive dose of an immunostimulant is applied topically.

60. The method of claim 39, wherein said one or several consecutive dose of an immunostimulant is applied topically.

61. The method of claim 40, wherein said one or several consecutive dose of an immunostimulant is applied topically.

62. The method of claim 41, wherein said one or several consecutive dose of an immunostimulant is applied topically.

63. The method of claim 42, wherein said one or several consecutive dose of an immunostimulant is applied topically.

64. The method of claim 43, wherein said one or several consecutive dose of an immunostimulant is applied topically.

65. The method of claim 44, wherein said one or several consecutive dose of an immunostimulant is applied topically.

66. The method of claim 45, wherein said one or several consecutive dose of an immunostimulant is applied topically.

67. The method of claim 46, wherein said one or several consecutive dose of an immunostimulant is applied topically.
